(19) United States
(12) Patent Application Publication
(10) Pub. No.: US 2022/0040917 A1
(43) Pub. Date: Feb. 10, 2022

(54) PROCESS FOR THE PREPARATION OF A COATED SOLID PHARMACEUTICAL DOSAGE FORM

(71) Applicant: MERCK PATENT GMBH, DARMSTADT (DE)

(72) Inventors: Malte BOGDAHN, Darmstadt (DE); Stefan SCHILLER, Mainz (DE); Andrea HANEFELD, Seeheim-Jugenheim (DE); Simon GEISSLER, Bad Homburg (DE)

(73) Assignee: MERCK PATENT GMBH, DARMSTADT (DE)

(21) Appl. No.: 17/275,770

(22) PCT Filed: Sep. 12, 2019
(86) PCT No.: PCT/EP2019/074296

§ 371 Date: Mar. 12, 2021

(30) Foreign Application Priority Data
Sep. 14, 2018 (EP) 18194418.2

(51) Int. Cl. B29C 64/165; A61K 9/20; B33Y 10/00; B33Y 80/00; B33Y 70/00

(52) U.S. Cl. CPC B29C 64/165; A61K 9/2095; B29L 2031/753; B33Y 80/00; B33Y 70/00; B33Y 10/00

(57) ABSTRACT

The present invention is directed to a process for the preparation of a coated solid pharmaceutical dosage form using 3D printing technology.

The present invention is directed to a process for the preparation of a coated solid pharmaceutical dosage form using 3D printing technology.

3D printing/additive manufacturing (AM) of pharmaceutical dosage forms is getting more and more attention from academics and industry alike. Commonly applied techniques include Fused Deposition Modeling (FDM; e.g. Ultimaker) (Melocchi, 2015). In this process polymer strands, commonly known as filaments, are heated to a semi molten state. The soft material is deposited through a spatial controlled nozzle. After the deposition of one layer of the material, the distance between nozzle and partial formed object is increased and the next layer of the object is build. Powder Bed and Binder Jetting (Goole, 2016) based processes use thin layer of powder which particles are selectively solidified by means of a suitable binder fluid. The fluid is typically deposited in small droplets. After the printing process excess powder is removed from the object. Laser Sintering (SLS) (Basit, 2018) and Multi Jet Fusion (MJF) processes solidify the powder with irradiation. SLS uses a focused laser which is scanned over the powder bed to selectively heat powder particles, while MJF uses an IR absorber which is printed onto the powder bed before irradiation of the whole bed. Excess powder is removed after the print. Printed objects commonly have a rough surface due to limited spatial resolution, usually limited by the size of the filament (FDM) or the particle size of the used powder. A common practice for smoothening the surface of printed plastics (e.g. PLA) is a post-processing step after removal of the printed object from the build platform. Usually a solvent treatment is employed, e.g. aceton wipe or vapor.

Pharmaceutical dosage forms are printed by using FDM (Melocchi, 2015), Binder Jetting (Goole, 2016), Laser Sintering (Basit, 2018), Multi Jet Fusion.

Pharmaceuticals printed from established AM techniques typically have a much higher surface roughness than conventionally produced tablets (e.g. granulation, compression, coating) due to the limited accuracy of the printing processes. This may lead to poor acceptance by patients. While tableting leads to deformation of individual particles to form a smooth surface, the surface accuracy for AM techniques is typically limited by constraints of the technique itself or by an unacceptable increase in processing time. For FDM, the accuracy is typically limited by the diameter of the extrusion nozzle and layer height and higher accuracy usually means lower throughput. Typical nozzle sizes have a diameter from 0.25 mm to 0.8 mm. The accuracy of Binder Jetting and Laser Sintering is limited by the height of the applied layer and the size and morphology of the individual particles in the powder bed.

An attractive option to smoothen a rough surface of a solid pharmaceutical dosage form as it is created by 3D printing/AM is to apply to such dosage form a coating. Such a coating may also be used for color correction, to introduce additional functionalities, e.g. enteric properties, delayed or extended release, taste masking, reduction of friability/prevention of (hazardous) dust formation, a protective barrier against moisture etc.

Although it is in principle possible to apply a coating to a 3D printed solid pharmaceutical dosage by a subsequent coating step this wouldn't be an attractive approach as it would invalidate some of the main benefits of AM for pharmaceuticals (e.g. reduced number of unit operations, increased flexibility in time and scale).

An ideal process would combine printing and coating in a single additive manufacturing method.

When using FDM, the outer layer may be printed from a curable polymer which is then treated as in plastics printing. AM techniques using a powder bed and inkjet head may be adapted to print the shell layer by layer around the dosage form. As the printing occurs while the loose powder is still surrounding the dosage form, the coating layer would lead to powder sticking more or less loosely to the surface of the printed dosage form. This powder again leads to surface roughness and may detach during bulk handling or handling by the patient/care giver, leading to (hazardous) dust formation and exposure.

It was an object of the present invention to provide a process for the manufacturing of a coated solid pharmaceutical dosage form, wherein both the solid pharmaceutical dosage form and the coating are manufactured by using 3D printing/AM and which process is not associated with the problems as described.

A process that meets such criteria is made available by the present invention.

The present invention is directed to a process for the manufacture of a solid pharmaceutical administration form comprising an active ingredient comprising the steps

(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient and optionally a functional material across the manufacturing area to create a layer;
(f) causing the powder created in step (e) to adhere in a defined pattern;
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

The process can be run on a 3D printer composed of a pair of horizontal X-Y axes that are suspended over a vertical piston, providing control over three directions of motion and that is equipped with jet head as known from ink jet printing technology. Preferably the jet head comprises a multichannel nozzle that allows printing of multiple fluids successively or in parallel. For manufacture of solid pharmaceutical dosage form a powder is spread onto a mounting plate to create a powder bed, the fluid is precisely distributed over predefined areas of the powder bed through a jet head that is moved over the powder bed. A material that is jet printed to the powder bed is a material capable to coalesce. Such material is placed around the solid pharmaceutical dosage form and provides alone or together with the powder layer on which it was jet printed a coating surrounding the pharmaceutical dosage form after such material is caused to coalesce into a layer in a later step. Depending from the embodiment further materials are jet printed to the powder, such as binding material or fusible material, to provide coherence and/or fusion of the powder to a solid dosage form, optionally after activation, e.g. by irradiation. After lowering the mounting plate by a fixed distance, a layer of powder is spread, and the process is repeated. Instead of lowering the mounting plate the spreading means can be raised by a fixed distance. In some instances, the process is run under elevated temperature (e.g. the build chamber is heated to above room temperature and below 100° C., preferably 30-60° C.) to improve processability, repeatability and evaporation of printing fluids. The heating can be applied during the whole process or parts of it, e.g. from before step (a) to after step (k). It may be advantageous to wait for the temperature in the build chamber to stabilize before continuing with the process.

As used herein, “a” or “an” shall mean one or more. As used herein when used in conjunction with the word “comprising,” the words “a” or “an” mean one or more than one. As used herein “another” means at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−1-3% of the recited value) that one of ordinary skill in the art would consider equivalent to the recited value (e.g., having the same function or result). In some instances, the term “about” may include numerical values that are rounded to the nearest significant figure.

The term “solid pharmaceutical administration form” as used herein means any pharmaceutical formulation that is solid and provides a dosage unit of an active pharmaceutical ingredient that can be administered to a patient by any way of application such as oral, rectal, vaginal, implantation. The solid pharmaceutical administration form can have any shape adapted to the application requirements, e.g. round, oval, rod like, torpedo shaped etc. Examples of solid pharmaceutical administration forms are tablets, pills, caplets, suppositories, implants.

The term “active ingredient” as used herein means any ingredient that provides a pharmacological or biological effect when applied to a biological system. The active ingredient may be a pharmaceutical drug, biological matter of viral or ling origin. Examples of an active ingredient that may be used in the process of the present inventions are insulin, heparin, calcitonin, hydrocortisone, prednisone, budesonide, methotrexate, mesalazine, sulfasalazine, amphotericin B, nucleic acids, or antigens (peptides, proteins, sugars, or other substances that form surfaces recognized by the immune system, either produced, extracted, or homogenized from tissue, an organism or a virus).

The term “spreading” as used herein means a process where a planar layer of powder is applied to a planar ground. Spreading of powder can be achieved by using means that are suitable to create a planar layer of powder. Examples of such means are a doctor blade or a roller that can be moved in parallel to planar ground such as a mounting area or an existing powder layer to distribute the powder from a reservoir across the planar ground. By the use of a roller a certain level of compaction can be obtained, which may be advantageous for the manufacture of the solid pharmaceutical dosage form.

The term “functional material” as used herein means any material that is not an active ingredient but is processible in AM processes and that provides mass and structure to the pharmaceutical dosage form. Depending from the specific process used, such as Binder Jetting, Fused Deposition Modelling or Multi Jet Fusion the functional material can have different properties that are suitable and/or necessary for running the respective process and to build up a pharmaceutical dosage form that meets the requirements to be fulfilled (e.g. disintegration time, dissolution or storage stability).

The term “jet printing” as used herein refers to a process where a fluid is distributed to the powder bed by ejecting droplets of fluid at high speed towards and onto the powder bed. Ejection of droplets can be performed with utmost precision to predefined target place. By managing size of droplets, the amount of droplets and the specific target place, the exact placement on and/or penetration depth in a substrate can be precisely controlled. Jet printing is well-known from inkjet printing technology but in contrast to this technology the fluid that is printed in the process of the present invention is not an ink for printing of images but a fluid that contains materials that are usable for printing of solid pharmaceutical administration forms, especially a material capable to coalesce, an energy absorbing or a reflecting material, a fusible material or an active ingredient.

The fluid used for jet printing comprises a liquid wherein the material to be printed is distributed. Examples of liquids that can be used for distribution of the material are water, organic solvents, such as ethanol, or mixtures of both, whereby the organic solvent may be soluble with one another or not. The material may be dissolved, suspended or emulsified in the fluid. Auxiliaries such as surfactants may be used, e.g. to improve dispersibility of the material in the fluid and/or spreading or wetting of particles in the powder bed.

In an alternative embodiment the material to be printed itself is a fluid when it is jet printed and converts to a solid or highly viscous after it is printed to the powder. A material usable for such embodiment is a material that is solid or highly viscous at room temperature but a fluid at elevated temperature (40-120° C., preferably 40-80° C. and more preferably 45-60° C.). In the process the material is heated so that it melts and is converted to a fluid prior to printing. One material or a mixture of materials can be used. Advantageously, jet printing of fluids prepared by melting the material to be printed to the powder does not require a liquid so that no liquid has to be removed afterwards. Examples of usable materials that can be used without a liquid are poly(oxy ethylene)s, poly(oxy propylene)s and their co-polymers.

The term “material capable to coalesce” means a material that is solid at room temperature that softens and gets flowable once the temperature is elevated above a certain value (40-120° C., preferably 50-90° C. and more preferably 50-60° C.) thereby causing such material to coalesce and to form a unit having a uniform surface. In the process of the present invention the material capable to coalesce is dissolved and/or dispersed in a fluid and jet printed onto of the existing layer so that the material capable to coalesce is distributed on the surface of the particles of existing layer. Once the fluid of the jet printed droplets is evaporated the material capable to coalesce remains on the surface of existing layer created prior to jet printing, which material is caused to coalesce into a layer in a later step of the process.

After jet printing a material capable to coalesce to the particles of existing layer (e.g. steps (c), (g) and (j)) the amount of such material applied to the particle might not be sufficient to create a uniform layer having the desired properties such as thickness and impermeability after the material was caused to coalesce. In such instances the spreading step preceding the jet printing step as well as the jet printing step may be repeated as often as needed to create a coating having desired properties (e.g. steps (d), (h), (k)).

The term “coating” as used herein refers to a layer on the surface of the solid pharmaceutical dosage form. In the process of the present invention the coating is provided by the material capable to coalesce that after being caused to coalesce forms a uniform layer on the surface of the solid pharmaceutical dosage form.

In steps (b) to (d) one or more layers are applied to the powder bed that build up the bottom of coating of the pharmaceutical dosage form. The term “bottom” as used herein refers to the position of the layer of the solid pharmaceutical dosage form with respect to the printer at the time of its manufacturing only and is independent from the geometry of the solid pharmaceutical dosage form. Accordingly, if, for example, a flat tablet with a cylindrical shape is manufactured and one flat side of such tablet is produced first, the term bottom refers to such flat side. Likewise, if the lateral edge of such flat tablet is produced first, the term bottom refers to such lateral edge of the tablet.

The core of the solid pharmaceutical dosage form is build up by performing steps (e) and (f). The term “core” as used herein refers to the solid pharmaceutical dosage form without a coating layer.

The term “upper side” as used herein refers to the position of the layer of the solid pharmaceutical dosage form with respect to the printer at the time of its manufacturing only and is independent from the geometry of the solid pharmaceutical dosage form. The upper side of the solid pharmaceutical dosage is located opposite to bottom of the pharmaceutical dosage form.

The material capable to coalesce is caused to coalesce by using appropriate measures such as irradiation, heating, moisture or vapor of water or organic solvent.

After causing the material capable to coalesce to coalesce into a layer (step (n)) the coated solid pharmaceutical administration form is prepared. In some instances, some powder from the manufacturing may remain loosely adhered at the solid pharmaceutical dosage form. In such instances the adhered powder is removed from the solid pharmaceutical dosage form in an additional step. Accordingly, the present invention is also directed to a process for manufacture of a solid pharmaceutical dosage form, that further comprises the step (o) removing loosely adhering powder from the solid pharmaceutical dosage form. Removing of adhering powder may be performed by appropriate methods such as blowing away using airflow and/or shaking.

The process of the invention allows the manufacture of a coated solid pharmaceutical dosage form using 3D printing technology. Depending from the method used for causing the active ingredient and/or functional material to adhere in a defined pattern the material and/or technical steps for performing this are different.

Process according to claim 1 or 2, wherein the powder in step (e) comprises a fusible material and wherein step (f) is performed by irradiation.

One appropriate method that can be used in the process of the present invention is the Fusion technology as described in WO 2018/046642 A1. In such method a powder comprising a fusible material is spread across the manufacturing area and the powder is irradiated. By irradiation the powder bed is heated which causes at least partial fusing of the fusible material and leads to adherence of the powder in a defined pattern.

Accordingly, the invention is also directed to the process as described herein, wherein the powder in step (e) comprises a fusible material and wherein step (f) is performed by irradiation.

The term “fusible material” is a material that melts and fuses upon heating. The fusible material has a rather low melting point or glass transition temperature to keep the operation temperature low and to keep potential detrimental effects on the solid pharmaceutical dosage form, especially the active ingredient, as low as possible but it has to be high enough to assure stability of the shape of the solid pharmaceutical dosage form under usual storage conditions, e.g. room temperature. Preferably, the glass transition temperature would be at least 20° C. higher than the projected storage condition at the same humidity. A suitable range of melting points or glass transition temperatures would be 50-150° C., more preferably 50-100° C., most preferably 60-80° C. Examples of fusible materials are lipids, incl. fats and waxes, derivatives thereof; resins; low melting sugars and sugar alcohols, incl. fructose, sorbitol, xylitol; mixtures of these to reduce melting point; modified sugars such as sucrose esters, sorbitan esters; vitamin E TPGS; pharmaceutical polymers with or without plasticizer (incl. water) with sufficiently low melting point or glass transition temperature, incl. PEG/PEO, PEO esters and ethers, PVAc, PVP, PCL, Poloxamers, PVPVA, celluloses and derivatives thereof, poly-acrylates, poly-methacrylates, PLA, PLGA, gelatin, alginate, shellac, agar; composites, mixtures and blends thereof.

As used herein, “fusing” means complete fusing or partial fusing. As used herein “melting” means complete or partial melting.

In an advantageous embodiment of such Fusion technology an energy absorbing material is added to the powder to induce/increase heat development upon irradiation. Preferably such energy absorbing material is added after step (e) and prior to step (f) by use of jet printing. Accordingly, the invention is also directed to a process as described above, wherein the process after step (e) and prior to step (f) further comprises the step (e1) jet printing a fluid comprising an energy absorbing material onto the powder. Such embodiment is also known as Multi Jet Fusion technology and described in WO 2018/046642 A1.

The term “energy absorbing material” as used herein means any material that absorbs IR, NIR, VIS, UV or microwave irradiation and converts it to some extend to heat. In principle, any energy absorbing material can be used in the present invention. Energy absorbing materials that are especially suitable for the present invention are carbon black, pigments and anorganic salts, e.g. oxides and salts and alloys of iron, zinc, magnesium, aluminium or other metals, organic dyes and liquids (e.g. water). Certain energy absorbing materials may further possess the property to reflect or scatter radiation, which may lead to an improved heat distribution. Examples may include pigments of a certain particle shape and size, pigments with layered structures and interference pigments such as composites comprising silicate minerals (such as sheet silicate (phyllosilicate) minerals (mica) or potassium aluminium silicate) and oxides of titanium or iron (Candurin® pigments). The energy absorbing material can be used in any form and particle size that is processable and that provides heat generation and distribution suitable for running the process

The process as described above uses an energy absorbing material to provide the heat that is necessary for melting and fusing of the fusible material. However, depending on the melting point or glass transition temperature of the fusible material, the absorption spectrum of components of the powder bed and the amount of thermal energy provided by the irradiation, the irradiation alone can be sufficient to induce melting and fusing of the fusible material so that the addition of an energy absorbing material is not necessary. In this case jet printing of an energy absorbing material can be replaced by jet printing of the fusible material.

Irradiation of the powder bed either directly induces heating of the fusible material and/or induces heating of the energy absorbing material in the powder, both leading to that the fusible material is at least partially fused and the powder is caused to adhere in a defined pattern.

When Multi Jet Fusion method is used in the present invention the whole process for the manufacture of a solid pharmaceutical administration form comprising an active ingredient comprises the following steps:

(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient, a fusible material and optionally a functional material across the manufacturing area to create a layer;
(e1) jet printing a fluid comprising an energy absorbing material onto the powder;
(f) irradiating the powder to cause adherence of the powder in a defined pattern;
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

In the process using Multi Jet Fusion method as described above the fusible material is introduced as part of the powder spread in step (e). In an alternative embodiment the fusible material is not introduced as part of the powder spread in step (e) but instead jet printed to the powder bed in step (e2). Accordingly, the present invention is also directed to a process, wherein the process after step (e) and prior to step (f) further comprises step (e2) jet printing a fluid comprising a fusible material and an energy absorbing material onto the powder and wherein step (f) is performed by irradiation. In step (e2) the energy absorbing material and the fusible material can jet printed from one fluid or from separate fluids. In the first instance the energy absorbing material and fusible material are combined in one fluid whereas in second instance the energy absorbing material is present in one fluid and the fusible material in another fluid. If more than one fluids are jet printed such fluids can be jet printed in parallel or subsequently.

Putting all steps together the whole process for the manufacture of a solid pharmaceutical administration form comprising an active ingredient comprises the following steps:

(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient, and optionally a functional material across the manufacturing area to create a layer;
(e2) jet printing a fluid comprising a fusible material and an energy absorbing material onto the powder;
(f) irradiating the powder to cause adherence of the powder in a defined pattern;
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

In some instances, the processes described above may lead to physical changes such as melting of the fusible material in places adjacent to the intended region. Especially processes using materials with broader melting or glass transition ranges or strong heat dissipation may be affected by this phenomenon. Such processes may be improved by selectively cooling of the fusible material in places adjacent to the intended region. Such improvement may be achieved by using a parting agent. As used herein a “parting agent” refers to an agent that facilitates the shape and removal of the object of fused powder created by the irradiation by minimizing or avoiding sticking of powder of the surrounding powder bed to the object. Minimizing or avoiding of powder sticking to the object can be achieved by selective cooling of the surrounding powder bed, preferably by evaporation cooling. Agents that may be used as parting agent comprise volatile fluids, preferably pharmaceutically acceptable solvents such as water, methanol or ethanol, liquid alkanes such as pentane, hexane or heptane, more preferably water or ethanol.

By precise placement of the parting agent to the intended edges in the powder bed shape accuracy and edge definition of the printed object can be improved. The parting agent may further serve as means to modulate surface or matrix porosity of the resulting dosage form.

In the process of the present invention precise placement of the parting agent can be easily realized by jet printing of the parting agent onto the powder, either in parallel or subsequently in step (e1) or step (e2). Accordingly, the invention is also directed to the process for the manufacture of a solid pharmaceutical administration form as set forth above, wherein in step (e1) or (e2) a parting agent is jet printed onto the powder in parallel or subsequently.

In some instances, the speed of the process described above may be improved by applying heat to the build chamber, the powder bed or powder supply with a suitable method without disrupting the powder bed. The powder may be heated to a temperature 2-50° C. below the melting point or glass transition temperature of the fusible material at which the powder bed still retains favorable flow properties. Accordingly, the invention is also directed to a process for the manufacture of a solid pharmaceutical administration form as set forth above, wherein heat is applied in steps (a), (b), (e) and/or (i) prior to and/or after spreading the powder.

In some instances, the process described above may lead to so much heat development that direct removal of the solid pharmaceutical dosage form from the powder bed after its manufacture causes damage of the solid pharmaceutical dosage form, especially damage of its shape. In this case a cooling step is introduced into the process. If needed such cooling step can be introduced at any stage of the process and may be run in parallel or between any of the process steps as defined. Preferably a cooling step is introduced after manufacturing of the solid pharmaceutical dosage form prior to its removal from the mounting plate, i.e. prior to step (l). Accordingly, the invention is also directed to a process for the manufacture of a solid pharmaceutical administration form as set forth above, wherein a cooling step is introduced at any stage of the process, preferably prior to step (l).

The cooling step comprises any method that leads to sufficient reduction of the temperature of the solid pharmaceutical form to a temperature value to assure that the shape of the solid pharmaceutical dosage form is maintained when it is removed from the mounting plate. Examples of a cooling step are simple remaining of the solid pharmaceutical dosage form on the mounting plate at ambient temperature until obtaining sufficient temperature reduction or active cooling, such as cooling by a cold air flow. Preferably, the cooling step would allow to control the cooling rate and thus the physical characteristics of the quenched melt.

The source of irradiation used in the process can be infrared energy (IR), near-infrared energy (NIR), visible light (VIS), ultraviolet light (UV), microwave or X-radiation. Infrared energy is preferred. The source of irradiation used in the process can be diffuse (e.g. lamps, gas discharge tubes) or focused (e.g. lasers). Therefore, the invention is further directed to a process that is characterized in that the irradiation is infrared energy (IR), near-infrared energy (NIR), visible light (VIS), ultraviolet light (UV), microwave or X-radiation, preferably infrared energy (IR). Therefore, the present invention is also directed to a process, wherein the irradiation energy is infrared energy (IR), near-infrared energy (NIR), visible light (VIS), ultraviolet light (UV), microwave or X-radiation, preferably IR.

Another appropriate method that can be used in the process of the present invention for causing the active ingredient and/or functional material to adhere in a defined pattern is Binder Jetting.

In such method a powder is spread across the manufacturing area and a fluid comprising a binding material is jet printed onto the powder. Accordingly, the invention is also directed to the process as described herein, wherein step (f) is performed by jet printing a fluid comprising a binding material onto the layer created by step (e).

The term “binding material” as used herein refers to any substance that is capable to provide adherence and cohesion to the powder and thereby to transform the powder to a solid when a fluid comprising the binding material is jet printed to the powder. Suitable binding materials are, for example, polymers, such as, for example, polyvinylpyrrolidone or polyvinyl acetate, starch such as, for example, maize starch, cellulose derivatives, such as, for example, hydroxypropylmethylcellulose or hydroxypropylcellulose.

Putting all steps together the whole process for the manufacture of a solid pharmaceutical administration form comprising an active ingredient comprises the following steps:

(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient, and optionally a functional material across the manufacturing area to create a layer;
(f) jet printing a fluid comprising a binding material onto the layer created by step (e);
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

After jet printing of the fluid comprising a binding material at least a part of the volatile material deposited on the powder layer is evaporated. While the volatile material present in the fluid is evaporated, the binding material remains on the powder and causes the powder to adhere in a defined pattern. Evaporation begins directly after the jet printing step (f) and lasts at least to prior to performing step (i). The same is true for printing of the fluid comprising a material capable to coalesce. Evaporation begins directly after the jet printing steps (c), (g), (j).

In some instances, the jet printed fluid is actively caused to evaporate to increase the extend/completeness of evaporation. The jet printed fluid may be actively caused to evaporate at any stage of the process beginning at the end of step (c) and ending before step (n). Accordingly, the present invention is also directed to process as described herein, wherein a jet printed fluid is actively caused to evaporate at any stage of the process beginning at the end of step (c) and ending before step (n). Measures to actively cause evaporation involve all appropriate measures such as heating and/or reducing of the atmospheric pressure.

In Binder Jetting the binding material is introduced to the powder as part of a fluid. In an alternative method introducing of a binding material and of a fluid are separated from each other. In such method the binding agent is part of the powder to be spread across the manufacturing area and a fluid capable to induce binding of the binding material is jet printed onto the powder. Accordingly, the invention is further directed to the process as described herein, wherein in step (e) the powder comprises a binding material and wherein step (f) is performed by jet printing a fluid capable to induce binding of the binding material onto the layer created by step (e).

The term “fluid capable to induce binding of the binding material” as used herein refers to any fluid that after being jet printed to a powder comprising a binding material induces binding of the binding material that is present in the powder and thereby causes the powder to adhere in a defined pattern. Suitable fluids are, for example, pharmaceutically acceptable solvents such as water and alcohols, e.g. ethanol or methanol and the like as well as mixtures thereof.

Compared to Binder Jetting the alternative method has several advantages. Firstly, as the binding material is uniformly distributed throughout the whole powder layer adherence is provided throughout the whole powder layer. In Binder Jetting the binding material is jet printed to the top of the powder layer so that uniform distribution of the binding material is not always ensured. Secondly, the amount of fluid to be jet printed is limited to an amount that is necessary to induce binding. Accordingly, the amount of volatile material, that has to be evaporated, can be kept at a minimum, and a drying step, if necessary at all, is shortened and/or can be run under milder conditions (e.g. under less heat or less decrease of atmospheric pressure). Binder Jetting by contrast uses fluid in larger quantities as the fluid is also needed as a carrier for the binding material.

However, in some instances it may be also necessary to actively cause the fluid to evaporate as described for the process involving Binder Jetting. Accordingly, the present invention is also directed to process as described herein, wherein the fluid jet printed in step (f) is actively caused to evaporate at any stage of the process beginning at the end of step (f) and ending before step (n), preferably before step (j).

Putting all steps together the whole process for the manufacture of a solid pharmaceutical administration form comprising an active ingredient comprises the following steps:

(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient, a binding material and optionally a functional material across the manufacturing area to create a layer;
(f) jet printing a fluid capable to induce binding of the binding material onto the layer created by step (e);
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

In all embodiments of the process described herein the material capable to coalesce is placed adjacent to core so that the core is completely surrounded by a material capable to coalesce. To achieve a uniform surrounding the material capable to coalesce is caused to coalesce into a layer. In an advantageous embodiment of the invention the material capable to coalesce is jet printed in a shape with a differing spatial thickness distribution as this reduces the amount of adhered powder on the exterior surface of the coating after being coalesced. Without being bound on this theory it is speculated that this effect is based on an increased surface tension of such an arrangement which reduces the physical adherence of the powder and/or on an increased internalization of such particles during coalescence. Accordingly, the present invention is also directed to a process as disclosed herein, wherein the material capable to coalesce is jet printed in a shape with a differing spatial thickness distribution.

Suitable materials that can be used as material capable to coalesce are polyethylene glycol/polyethylene oxide (PEG/PEO), polyethylene oxide esters and ethers, poloxamers, polyvinyl alcohol (PVA), polyvinyl acetate (PVAc), polyvinylpyrrolidone (PVP), polyvinylpyrrolidon/vinylacetate copolymers (PVP/VA), polycaprolactone (PCL), cellulose and its derivatives, such as hydroxypropyl methylcellulose (HPMC), hydroxyethyl cellulose (HEC), ethyl cellulose (EC), hydroxypropyl cellulose (HPC), hydroxypropyl methylcellulose phthalate (HPMCP), hydroxypropyl methylcellulose acetate succinate (HPMCAS), acrylic and methacrylic polymers, waxes, polylactic acid (PLA), poly(lactic-co-glycolic acid) (PLGA), gelatin, alginate, shellac, agar, composites, mixtures and blends thereof. Accordingly, the present invention is further directed to a process, wherein the material capable to coalesce is polyethylene glycol/polyethylene oxide (PEG/PEO), polyethylene oxide esters and ethers, poloxamers, polyvinyl alcohol (PVA) polyvinyl acetate (PVAc), polyvinylpyrrolidone (PVP), polyvinylpyrrolidon/vinylacetate copolymers (PVP/VA), polycaprolactone (PCL), cellulose and its derivatives, such as hydroxypropyl methylcellulose (HPMC), hydroxyethyl cellulose (HEC), ethyl cellulose (EC), hydroxypropyl cellulose (HPC), hydroxypropyl methylcellulose phthalate (HPMCP), hydroxypropyl methylcellulose acetate succinate (HPMCAS), acrylic and methacrylic polymers, waxes, polylactic acid (PLA), poly(lactic-co-glycolic acid) (PLGA), gelatin, alginate, shellac, agar, composites, mixtures and blends thereof.

In some instances, a material that facilitates coalescence of the material capable to coalesce is added to improve the formation of the layer, the homogeneity of the layer and/or the uniformity of the coating thickness. Suitable materials that facilitates coalescence are, for example, a plasticizer, an energy absorbing material and/or a surfactant. Accordingly, the invention is also directed to a process as described herein, wherein the fluid used in steps (c), (g) and/or (j) comprises a material that facilitates coalescence of the material capable to coalesce such as a plasticizer, an energy absorbing material and/or a surfactant. Suitable plasticizers are additives that lower the melting point or glass transition temperature (by 5° C. or more), increase the plasticity or decrease the viscosity of a material capable to coalesce. Examples for plasticizers are polyethylene glycol/polyethylene oxide (PEG/PEO), polyethylene oxide esters and ethers, Poloxamers, glycerol, esters of polyols (e.g. glycerol, monoglycerides) or polycarboxylic acids (e.g. citric acid) such as triacetin, triethyl citrate, tributyl citrate. Suitable surfactants include polyethoxylated castor oil, ethoxylated soribitans, sorbitan fatty acid esters, ethoxylated sorbitol and sorbitol esters, ethoxylated fatty acids, polyethylene glycol fatty acids esters, ethoxylated alcohols and ethoxylated triglycerides, alkyl esters or salts of carboxylic acids (e.g. sodium dodecyl sulfate, sodium stearate), macrogol glycerol ethers. If an energy absorbing material is present, causing coalescence of the material to coalesce involves irradiation of the solid pharmaceutical dosage form.

In some instances, the material capable to coalesce further includes a material that improves the appearance such as a colouring agent and/or a pigment.

Coalescence of the material capable to coalesce can be caused by various measures such as irradiation, heat, moisture, or a vapor of water or organic solvent. While irradiation and heat induces coalescence by increase of temperature moisture and vapor facilitate coalescence by partial dissolution or softening of the surface. Accordingly, the present invention is as well directed to a process, wherein the coalescence in step (n) is performed by irradiation, heat, moisture, or a vapor of water or organic solvent.

The invention is illustrated in the Figures.

FIG. 1 illustrate the spreading step (a) of the process. Onto a mounting plate (1) a powder provided by a powder reservoir (3a) is spread by moving a doctor blade (4) in the direction indicated by an arrow to achieve a powder layer. A part of the powder layer that is already spread is indicated by (3). By repeating of the spreading of powder on the already existing powder layer(s) as often as necessary a powder bed (2) is created. Spreading of powder in other steps (e.g. steps (b), (e), (i)) is performed in the same way.

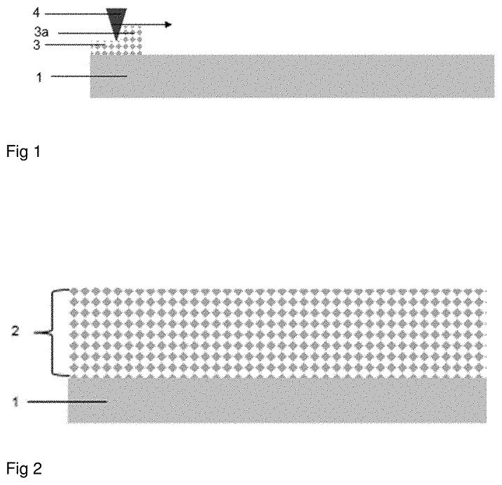

FIG. 2 shows the powder bed (2) that is created by step (b) on the mounting plate.

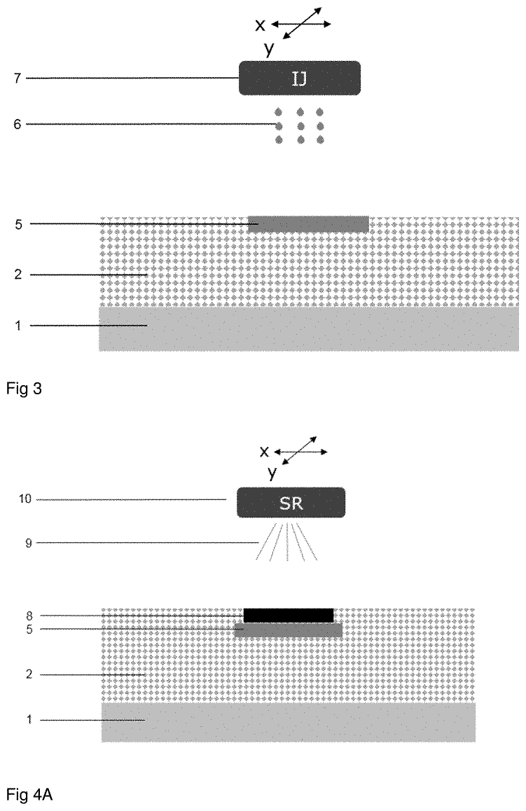

FIG. 3 shows jet printing in accordance to step (c) of the process. An inkjet head (IJ) (7) is moved along x and/or y axis thereby jet printing a fluid (6) comprising a material capable to coalesce (in fine droplets) onto the powder bed (2). Such jet printing results in powder soaked with fluid (5) created by voxels that are adjacent to one another. From the IJ (7) more than one fluid can be jet printed subsequently and/or in parallel depending on the process.

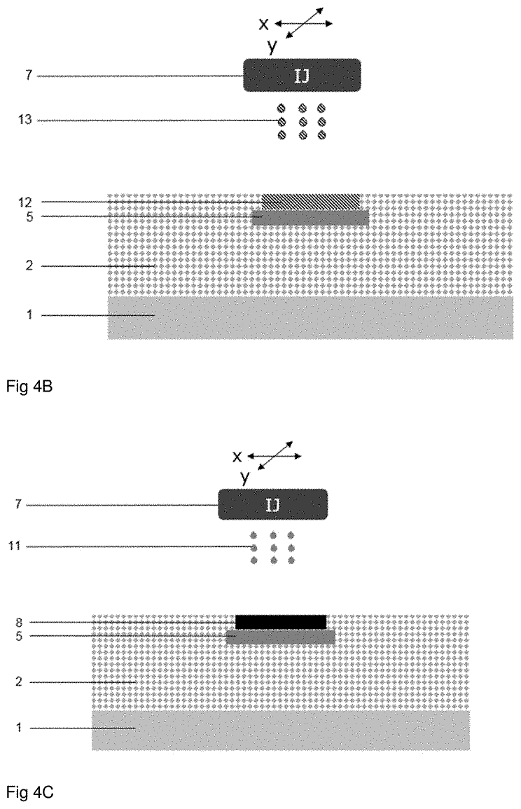

FIG. 4A shows one embodiment of step (f) wherein irradiation is used to cause the powder to adhere in a defined pattern. A source of radiation (SR) (10) is moved along x and/or y axis above the powder layer comprising a fusible material created in step (e). Upon irradiation (9) by the SR the fusible material present in the powder fuses thereby creating a layer of fused powder (8).

FIG. 4B shows a variation of the embodiment of the process shown in FIG. 4A wherein prior to irradiation step shown in FIG. 4A a fluid comprising an energy absorbing material (13) is jet printed to the powder layer comprising a fusible material.

FIG. 4C shows another embodiment of step (f) wherein Binder Jetting is used to cause the powder to adhere in a defined pattern. An inkjet head (IJ) (7) is moved along x and/or y axis thereby jet printing a fluid comprising a binding material (11) (in fine droplets) onto the powder layer that was spread on top of the layer soaked with the fluid comprising the material capable coalesce (5) thereby creating a layer of solidified powder (8).

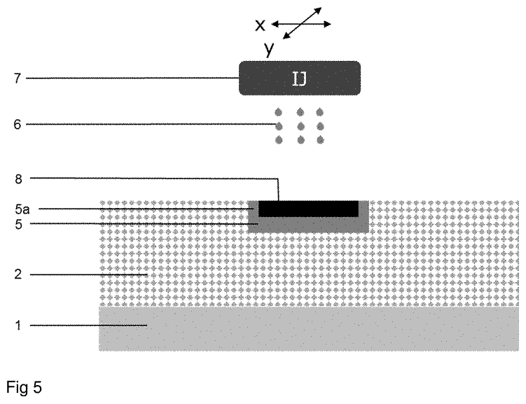

FIG. 5 shows jet printing in accordance to step (g) of the process. An inkjet head (IJ) (7) is moved along x and/or y axis thereby jet printing a fluid (6) comprising a material capable to coalesce (in fine droplets) around and adjacent to the shape of the solidified powder (8) onto the powder bed (2). Such jet printing results in powder soaked with fluid (5a) created by voxels that are adjacent to one another.

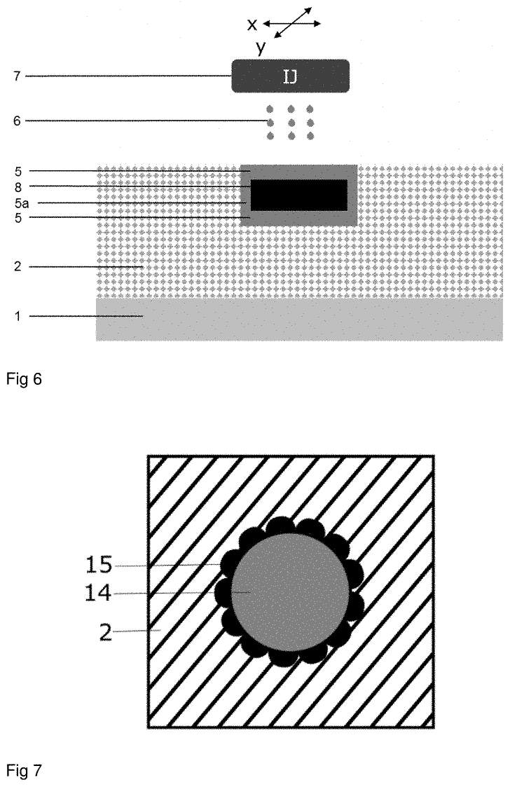

FIG. 6 shows jet printing in accordance to step (j) of the process. An inkjet head (IJ) (7) is moved along x and/or y axis thereby jet printing a fluid (6) comprising a material capable to coalesce (in fine droplets) onto the powder bed (2). Such jet printing results in powder soaked with fluid (5) created by voxels that are adjacent to one another.

FIG. 7 shows a section view of an embodiment of the solid pharmaceutical dosage form within a powder bed (2) wherein the material capable to coalesce is jet printed around and adjacent to the core (14) in a shape with a differing spatial thickness distribution (15).

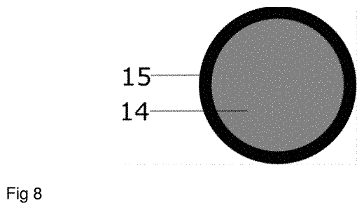

FIG. 8 shows the pharmaceutical dosage form as shown in FIG. 7 after it has been removed from the powder bed (step (m)) and the material capable to coalesce has been caused to coalesce into a uniform layer (15).

1. A process for the manufacture of a solid pharmaceutical administration form comprising an active ingredient comprising the steps
(a) spreading a powder comprising a functional material and/or an active ingredient across the manufacturing area to create a powder bed;
(b) spreading a powder comprising a functional material and/or an active ingredient across the manufacturing area to create a layer;
(c) jet printing a fluid comprising a material capable to coalesce onto the layer created by step (b);
(d) optionally repeating steps (b) and (c) as often as needed to build up the coating on the bottom of the solid pharmaceutical administration form;
(e) spreading a powder comprising an active ingredient and optionally a functional material across the manufacturing area to create a layer;
(f) causing the powder layer created in step (e) to adhere in a defined pattern;
(g) jet printing a fluid, comprising a material capable to coalesce, around and adjacent to the shape of the pattern defined in (f);
(h) optionally repeating steps (e) to (g) as often as needed to build up the core of the solid pharmaceutical administration form;
(i) spreading a powder comprising a functional material and/or an active ingredient across the manufacturing area to create a layer;
(j) jet printing a fluid comprising a material capable to coalesce onto the layer created by step (i);
(k) optionally repeating steps (i) and (j) as often as needed to build up the coating on the upper side of the solid pharmaceutical administration form;
(l) separating the solid pharmaceutical administration form from the powder bed;
(m) optionally removing loosely adhering powder from the solid pharmaceutical dosage form;
(n) causing the material capable to coalesce to coalesce into a layer.

2. Process according to claim 1, further comprising the step
(o) removing loosely adhering powder from the solid pharmaceutical dosage form.

3. Process according to claim 1, wherein the powder in step (e) comprises a fusible material and wherein step (f) is performed by irradiation.

4. Process according to claim 3, wherein the process after step (e) and prior to step (f) further comprises the step (e1) jet printing a fluid comprising an energy absorbing material onto the powder.

5. Process according to claim 1, wherein the process after step (e) and prior to step (f) further comprises the step (e2) jet printing a fluid comprising a fusible material and an energy absorbing material onto the powder and wherein step (f) is performed by irradiation.

6. A process according to claim 4, wherein in step (e1) or (e2) a parting agent is jet printed onto the powder in parallel or subsequently.

7. A process according to claim 1, wherein heat is applied in steps (a), (b), (e) and/or (i) prior to and/or after spreading the powder.

8. Process according to claim 1, wherein a cooling step is introduced at any stage of the process, preferably prior to step (l).

9. Process according to claim 3, wherein the irradiation energy is infrared energy (IR), near-infrared energy (NIR), visible light (VIS), ultraviolet light (UV), microwave or X-radiation, preferably IR.

10. Process according to claim 1, wherein step (f) is performed by jet printing a fluid comprising a binding material onto the layer created by step (e).

11. Process according to claim 1, wherein in step (e) the powder comprises a binding material and wherein step (f) is performed by jet printing a fluid capable to induce binding of the binding material onto the layer created by step (e).

12. Process according to claim 1, wherein a jet printed fluid is actively caused to evaporate at any stage of the process beginning at the end of step (c) and ending before step (n).

13. Process according to claim 1, wherein the material capable to coalesce is printed in a shape with a differing spatial thickness distribution.

14. Process according to claim 1, wherein the material capable to coalesce is polyethylene glycol/polyethylene oxide (PEG/PEO), polyethylene oxide esters and ethers, poloxamers, polyvinyl alcohol (PVA) polyvinyl acetate (PVAc), polyvinylpyrrolidone (PVP), polyvinylpyrrolidon/vinylacetate copolymers (PVP/VA), polycaprolactone (PCL), cellulose and its derivatives, such as hydroxypropyl methylcellulose (HPMC), hydroxyethyl cellulose (HEC), ethyl cellulose (EC), hydroxypropyl cellulose (HPC), hydroxypropyl methylcellulose phthalate (HPMCP), hydroxypropyl methylcellulose acetate succinate (HPMCAS), acrylic and methacrylic polymers, waxes, polylactic acid (PLA), poly(lactic-co-glycolic acid) (PLGA), gelatin, alginate, shellac, agar, composites, mixtures and blends thereof.

15. Process according to claim 1, wherein the fluid used in steps (c), (g) and/or (j) comprises a material that facilitates coalescence of the material capable to coalesce such as a plasticizer, an energy absorbing material and/or a surfactant.

16. Process according to claim 15, wherein the plasticizer is polyethylene glycol/polyethylene oxide (PEG/PEO), polyethylene oxide esters and ethers, Poloxamers, glycerol, esters of polyols (e.g. glycerol, monoglycerides) or polycarboxylic acids (e.g. citric acid) such as triacetin, triethyl citrate, tributyl citrate, mixtures and blends thereof.

17. Process according to claim 15, wherein the surfactant is polyethoxylated castor oil, ethoxylated soribitans, sorbitan fatty acid esters, ethoxylated sorbitol and sorbitol esters, ethoxylated fatty acids, polyethylene glycol fatty acids esters, ethoxylated alcohols and ethoxylated triglycerides, alkyl esters or salts of carboxylic acids (e.g. sodium dodecyl sulfate, sodium stearate), macrogol glycerol ethers, mixtures and blends thereof.

18. Process according to claim 1, wherein the coalescence in step (n) is performed by irradiation, heat, moisture, or a vapor of water or organic solvent.
